(19) United States
(12) Patent Application Publication
(10) Pub. No.: US 2006/0073204 A1
(43) Pub. Date: Apr. 6, 2006

(54) Modified release oral pharmaceutical composition

(76) Inventors: Sandhya Goyal, Delhi (IN); Sudir Verma, New Delhi (IN); Harvinder Popli, New Delhi (IN)

Correspondence Address: Jayadeep R. Deshmukh;Ranbaxy Inc., 600 College Raod East, Suite 2100, Princeton, NJ, 08540, US

(21) Appl. No.: 10/506,476

(22) PCT Filed: Mar. 5, 2002
(86) PCT No.: PCT/IB02/00645

§ 371 Date: Jul. 19, 2005

(51) Int. Cl. A61K 9/24

(52) U.S. Cl. 424/471

(57) ABSTRACT

The present invention relates to a modified release oral pharmaceutical composition comprising a core containing an active ingredient; and a coating surrounding said core, wherein said coating comprises a dispersion of water insoluble hydrophilic and hydrophobic polymers in a pharmaceutically acceptable organic solvent, which prevents the release of the active ingredient in the gastric fluid.

### FIELD OF THE INVENTION

The present invention relates to a modified release oral pharmaceutical composition comprising a core containing an active ingredient; and a coating surrounding said core, wherein said coating comprises a dispersion of water insoluble hydrophilic and hydrophobic polymers in a pharmaceutically acceptable organic solvent, which prevents the release of the active ingredient in the gastric fluid.

### BACKGROUND OF THE INVENTION

Modified-release pharmaceutical compositions include compositions having delayed, sustained and targeted-release characteristics. With modified-release dosage form, the time and location of the drug release can be controlled. There are several prior art processes that describe the preparation of modified release pharmaceutical compositions using hydrophilic and hydrophobic polymers. However, these modified release drug deliveries either provide the targeted release and/or sustained release and therefore the drugs that have corrosive effect on stomach wall or are degraded by stomach acids/digestive enzymes need additional enteric coating.

U.S. Pat. No. 4,252,786 relates to a controlled release tablet having a core containing the active ingredient dispersed in a blend of polymeric polyvinylpyrrolidone and a carboxyvinyl hydrophilic polymer and coated with a relatively insoluble, water permeable, rupturable film comprising a combination of hydrophobic and hydrophilic polymers. The release rate of active ingredient is initially primarily controlled by the film and after rupture or erosion of the film the release rate is controlled by the compressed matrix. The release of the active ingredient through the film begins within about one hour after the tablet has been ingested.

This dosage form doesn't prevent the release of active ingredient in the stomach and hence may not be used for irritant drugs or drugs which are degraded in acidic pH.

U.S. Pat. No. 4,610,870 describes a controlled release coated tablet comprising a core, which contains active and one or more hydrocolloid gelling agents. The core is coated with a coating composition containing a hydrophobic polymer and a hydrophilic polymer. Upon ingestion the coating slowly peels off leaving the core contents in contact with the gastric fluid, which then hydrate and swell to form a gelatinous mass which acts as a protective barrier. The active is slowly released by diffusion or leaching through the gel layer of the core.

In this system, coating only serves to prevent the initial burst release of the drug, with core being mainly responsible for controlling the drug release. Moreover, the coating provides no enteric protection.

U.S. Pat. No. 4,891,223 relates to a composition having a controlled, sustained release delivery pattern when contacted with a suitable surrounding media. The composition comprises:
a pharmaceutically active material core, soluble in a given surrounding media, the core present in an amount at least sufficient for a total dosage during a treatment period;
a first coating enveloping the active material core comprising a polymer or a blend of polymers, said polymer or blend of polymers being swellable upon penetration by the surrounding media; and
a second coating enveloping the first coating enveloped active material core comprising a polymer or a blend of polymers; said polymer or blend of polymers being water-insoluble and forming a semi-permeable barrier permitting diffusion of the surrounding media into the first coating enveloped active material core and also permitting the diffusion of the surrounding media dissolved active material into the surrounding media.

Multiple coatings result in high production cost. Moreover, the release from these multiple coated systems may be variable.

U.S. Pat. No. 5,260,069 describes a pulsatile particulate drug delivery system. The pellets are composed of a core containing the drug and a swelling agent which expands when exposed to water. The core is enclosed within a membrane or coating which is permeable to water. The membrane is composed of a water-soluble polymer, water-insoluble polymer and a permeability reducing agent. When the unit dose releases the pellets into the digestive tract, water diffuses through the coating into the core. As the swelling agent takes up water, the core expands, exerting force on the coating until it bursts, releasing the drug. The permeability reducing agents reduces the rate at which water reaches the swelling agent, thereby delaying release time. The water-soluble polymer dissolves, weakening the coating so that it bursts sooner. By varying the proportions of the three coating ingredients and/or coating thickness from one pellet population to another, the release timing of the pellets can be controlled.

Though the above system is good for pulsatile delivery, continuous drug release is not possible with such a system; also it depends on the swelling ability of the core which may be a limiting factor in case of drugs with high doses.

U.S. Pat. No. 5,840,332 discloses a gastrointestinal drug delivery system comprising a core and a coating. The coating is composed of a water insoluble carrier with a water insoluble hydrophilic particulate matter embedded in it which acts as a channeling agent, and thereby produce an in-vitro dissolution rate faster than the coating comprising the water insoluble carrier only. It further discloses a process for the preparation of the coating suspension by dissolving the polymer in the solvent, preferably in ethanol and then adding the particulate matter. The suspension is continuously stirred.

The system does not provide an enteric protection. In cases where it is required, an additional enteric coat is needed. Moreover it requires a swellable core, which may be a limiting factor in case of high dose drugs. Also dispersing particulate matter in the solution of a polymer could be difficult as it may form lumps. The suspension used for coating requires vigorous stirring throughout the coating process, which besides consuming energy may also, result in foaming.

Our co-pending patent application WO 01/87269 relates to an extended release formulation. It discloses a coated drug-containing core, the coating being an aqueous coating comprising an aqueous polymer dispersion of a water insoluble film forming polymer in combination with an aqueous colloidal solution of a high viscosity swellable polymer. The coating of the above system does not prevent the release of the drug in the stomach. Moreover, aqueous coatings require high temperatures and are not suitable for thermolabile drugs.

In light of the foregoing, it is clear that none of the modified release drug delivery systems heretofore described provides satisfactory enteric protection to the drug.

### SUMMARY OF THE INVENTION

Accordingly, the object of the present invention is to provide a modified release oral pharmaceutical composition comprising

a) a core containing an active ingredient; and

b) a coating surrounding said core,

wherein said coating composition comprises a dispersion of an anionic water insoluble hydrophilic polymer and a water insoluble hydrophobic film forming polymer, in a pharmaceutically acceptable organic solvent which prevents the release of the active ingredient in the gastric fluid.

The coating may be modified to work as an enteric coat only or to act as a timed-release system. It doesn't require the presence of a swellable agent in the core for the controlled release.

This coating provides enteric protection to the dosage. The dosage form of the present invention does not release the drug in media simulating the gastric conditions but swells when placed in a media simulating the intestinal conditions. These swelled portions allow the entry of the water into the dosage form and subsequently the release of the drug at a controlled rate.

As no drug is released in gastric fluid, it may be easily used for the drugs which degrade in acidic pH and the drugs which have harmful effect on the gastric lining.

The core of the present invention may be a matrix tablet or a capsule containing the drug or pellets of the pure drug or pellets of the drug layered on a core material or microcapsules containing the drug material.

The water insoluble anionic hydrophilic polymer of the coating composition may be selected from the group consisting of polyacrylic acids such as carboxyvinyl polymer, carbopol and polycarbofil; gums such as guar gum, xanthan gum, tragacanth gum, carragenan, locust bean gum; alginates; pectins and their metallic salts. However, carboxyvinyl polymers marketed by “B F Goodrich” as Carbopols are preferred.

The water insoluble hydrophobic film-forming polymers may be selected from the group consisting of cellulose ethers, shellac, zein, and waxes. However, cellulose ethers such as ETHOCEL marketed by “Dow Chemical Company” such as Standard 7, 10, or 20 Premium are preferred.

The anionic hydrophilic polymer to hydrophobic film-forming polymer ratio may range from 1:9 to 9:1.

The coating composition may optionally contain a plasticizer depending upon the function the coating is intended to perform. For example, if the coating is to function as an enteric coating then plasticizer is not required. For targeting different parts of intestine different percentages of plasticizer may be added to attain the release at the desired site. The plasticizers may be selected from the group consisting of dibutyl phthalate, diethyl phthalate, dimethyl phthalate, benzyl benzoate, butyl and glycol esters of fatty acids, refined mineral oils, oleic acid, stearic acid, cetyl alcohol, stearyl alcohol, castor oil, corn oil, camphor, and mixtures thereof.

Pigments, colorants, antifoam agents, antioxidants, waxes, monoglycerides, emulsifiers, surfactants and other additives may be added to the dispersion either to adjust its viscosity or to modify the resultant film properties.

Any pharmaceutically acceptable organic solvent may be used for this coating composition and include those selected from the group consisting of isopropyl alcohol, ethanol, acetone, or mixtures thereof. Organic solvents may also be among themselves mixed with small amounts of water. However, isopropyl alcohol is the preferred solvent, especially for moisture sensitive drugs.

The presence of the water insoluble hydrophobic film forming polymer along with an anionic water insoluble hydrophilic polymer creates an environment, which prevents the swelling of the anionic water insoluble hydrophilic polymer in the acidic pH of gastric media, as a result no water reaches the core. However, when the system is placed in the intestinal media (alkaline pH), the anionic water insoluble hydrophilic polymer undergoes neutralization reaction with the basic moieties present in the media leading to the swelling. This swollen anionic hydrophilic polymer present at the surface causes the media to pass to anionic hydrophilic polymer present inside the coating, which continues till the media reaches the core. This results in the formation of the several zigzag pathways for the passage of the media from outside to inside of the system or device and passage of the drug from inside to the outside surrounding media.

The coating composition may be modified by changing the ratio of the anionic water insoluble hydrophilic polymer to that of the water insoluble hydrophobic film forming polymer used; amount of the plasticizers; thickness of the coating.

For the purpose of the present invention, the coating suspension is prepared by dispersing the anionic water insoluble hydrophilic polymer into the solvent and then dissolving the water insoluble hydrophobic film forming polymer into the dispersion of anionic water insoluble hydrophilic polymer. This results in the formation of a uniform dispersion, which may be stirred intermittently to maintain the uniformity throughout the coating process.

Most commonly used enteric polymers (eudragits) exhibit sticky behavior while coating and require large quantities (as high as 50% by weight of the polymer) of anti tack agent like talc which results in poor mechanical strength of the film. This may be overcome by applying higher percentage of coating but it results in longer coating process times and variable dissolution profiles because of the thick coating. The composition of the coating process disclosed herein does not require any such anti tack agents and also application rate may be fast. This results in tablets with thin enteric coat, which may be obtained easily and quickly.

The coating composition of the present invention may be used to coat active ingredients in a solid dosage form such as tablets, beads, granules or capsules having sufficient integrity and particle size by methodology known in the art. Typical coating methods for applying coating are fluidized bed and side vented pan-coating processes. In these processes, a coating formulation is applied via spray nozzles onto the dosage forms. The dosage form is fluidized with hot air or agitated in a rotating pan with heated air supply while applying the coating to prevent agglomeration and to dry the polymer film. Temperatures are kept between 30-40° C. Because of the low temperatures involved, it is possible to coat the cores containing temperature sensitive drugs. Both processes result in a uniform film being applied to the surface of the active ingredient.

### DETAILED DESCRIPTION OF THE INVENTION

Embodiments of this invention are illustrated, but in no way limited, by the following examples.

### EXAMPLE 1

Coating Composition:

| | |
|---|---|
| Ethyl cellulose | 63% |
| Carbopol | 37% |
| Isopropyl alcohol | q.s. |

Carbopol was added to isopropyl alcohol with stirring. Carbopol gets hydrated in isopropyl alcohol and forms dispersion. To this dispersion, ethylcellulose and castor oil were added. This dispersion of hydrated carbopol in the solution of ethylcellulose in isopropyl alcohol was then coated on the pravastatin sodium, diclofenac sodium, atorvastatin calcium, metronidazole benzoate, pioglitazone tablets using a spray gun in a hicoater.

The percentage drug release from these coated tablets was studied in 0.1N HCl followed by the release in 6.8 phosphate buffer. The drug release profile is summarized below in Tables 1-5.

TABLE 1
Drug release from coated Pravastatin sodium tablets*

| | % drug released w.r.t Time (hrs) | | | | | | |
|---|---|---|---|---|---|---|---|
| Media | 1 | 2 | 3 | 4 | 5 | 6 | 7 |
| 0.1 N HCl | 0.63 | 1.16 | — | — | — | — | — |
| Phosphate | — | — | 10.49 | — | 33.32 | 59.09 | 75.62 |
| buffer(pH 6.8) | | | | | | | |

*with 6% weight build up

TABLE 2
Drug release from coated Diclofenac sodium tablets*

| | % drug released w.r.t Time (hrs) | | | | | | |
|---|---|---|---|---|---|---|---|
| Media | 1 | 2 | 3 | 4 | 5 | 6 | 7 |
| 0.1 N HCl | 0.24 | 0.12 | — | — | — | — | — |
| Phosphate | — | — | 5.63 | 68.85 | 88.07 | 96.86 | — |
| buffer (pH 6.8) | | | | | | | |

*with 4% weight build up

TABLE 3
Drug release from coated Atorvastatin calcium tablets*

| | % drug released w.r.t Time (hrs) | | | | | | |
|---|---|---|---|---|---|---|---|
| Media | 1 | 2 | 3 | 4 | 5 | 6 | 7 |
| 0.1 N HCl | — | 1.84 | — | — | — | — | — |
| Phosphate | — | — | 72.59 | 91.59 | 97.59 | — | — |
| buffer(pH 6.8) | | | | | | | |

with 8% weight build up

TABLE 4
Drug release from coated Metronidazole benzoate tablets*

| | % drug released w.r.t Time (hrs) | | | | | | |
|---|---|---|---|---|---|---|---|
| Media | 1 | 2 | 3 | 4 | 5 | 6 | 7 |
| 0.1 N HCl | — | 0.38 | — | — | — | — | — |
| Phosphate | — | — | 60.94 | 73.88 | 84.13 | — | 95.48 |
| buffer(pH 6.8) | | | | | | | |

*with 8% weight build up

TABLE 5
Drug release from coated Pioglitazone tablets*

| | % drug released w.r.t Time (hrs) | | | | | | |
|---|---|---|---|---|---|---|---|
| Media | 1 | 2 | 3 | 4 | 5 | 6 | 7 |
| 0.1 N HCl | — | 0.38 | — | — | — | — | — |
| Phosphate | — | — | 60.94 | 73.88 | 84.13 | — | 95.48 |
| buffer(pH 6.8) | | | | | | | |

*with 6% weight build up

These tablets showed no release of the drug in the acidic media (0.1 N HCl) for three hours. However, disintegrated completely when the medium was changed to pH 6.8 Phosphate Buffer. This indicated that had Pioglitazone been a soluble drug, complete release would have been attained.

Examples-2 & 3 and their corresponding drug release profile show the effect of Polymer ratio on Diclofenac sodium tablets.

### EXAMPLE 2

Effect of Polymer Ratio on Diclofenac Sodium Tablets

Carbopol and ethylcellulose in a ratio of 9:1 and 1:9 were dispersed in isopropyl alcohol and coated on diclofenac tablets. The dissolution data on these tablets is shown in Table-6.

TABLE 6
Dissolution in diclofenac sodium tablets in 0.1N HCl (2 hours)
followed by pH 6.8 Phosphate buffer, 900 ml, 75 rpm

| Ratio of | | | | | | | | | | | |
|---|---|---|---|---|---|---|---|---|---|---|---|
| carbopol to | | % drug dissolved w.r.t time (hours) | | | | | | | | | |
| ethylcellulose | Media | 1 | 2 | 3 | 4 | 5 | 6 | 8 | 10 | 12 | 14 |
| 9:1 | 0.1N HCl | 0.23 | | | | | | | | | |
| | pH 6.8 | — | — | 46.83 | 78.32 | | | | | | |
| | Phosphate | | | | | | | | | | |
| | buffer | | | | | | | | | | |
| 1:9 | 0.1N HCl | 1.31 | 0.31 | | | | | | | | |
| | pH 6.8 | | | 1.16 | 2.35 | 7.35 | 11.52 | 32.69 | 53.41 | 73.71 | 86.05 |
| | Phosphate | | | | | | | | | | |
| | buffer | | | | | | | | | | |

### EXAMPLE 3

Effect of Plasticiser (Castor Oil) Concentration on Release Characteristics of Diclofenac Sodium Tablets

Castor oil in different concentrations i.e. 0, 6, 12, 18, 25% was added to a dispersion of Carbopol and ethylcellulose in a ratio of 3.7:6.3 in isopropyl alcohol and coated on diclofenac tablets. The dissolution data on these tablets is shown in Table-7.

TABLE 7
Dissolution of diclofenac sodium tablets in
Phosphate buffer, pH 6.8, 900 ml, 75 rpm

| % of | % drug dissolved w.r.t time (hours) | | |
|---|---|---|---|
| plasticizer | 1 | 2 | 3 |
| 0 | 6 | 69 | 93 |
| 6 | 9 | 85 | 92 |
| 12 | 0.3 | 35 | 85 |
| 18 | 14 | 29 | 47 |
| 25 | 0.3 | 4 | 10 |

* weight build up 6%

### EXAMPLE 4

Coating Composition Exhibiting Controlled Release Characteristics in Diclofenac Sodium Tablets.

Carbopol and ethylcellulose in a ratio of 3.7:6.3 were dispersed in isopropyl alcohol and castor oil 25% was added to it and coated on diclofenac tablets. The dissolution data on these tablets is shown in Table-8.

TABLE 8
Dissolution of diclofenac sodium tablets in
pH 6.8 Phosphate buffer, 900 ml, 75 rpm

| Time (Hours) | % Drug Dissolved |
|---|---|
| 1 | 7.02 |
| 2 | 23.59 |
| 4 | 38.95 |
| 6 | 53.41 |
| 8 | 63.50 |
| 10 | 67.50 |
| 12 | 71.71 |

* 4% Weight build up

While the present invention has been described in terms of its specific embodiments, certain modifications and equivalents will be apparent to those skilled in the art and are intended to be included within the scope of the present invention.

We claim:

1. A modified release oral pharmaceutical composition comprising:
a) a core containing an active ingredient; and
b) a coating surrounding said core,
wherein said coating composition comprises a dispersion of an anionic water insoluble hydrophilic polymer and a water insoluble hydrophobic film forming polymer in a pharmaceutically acceptable organic solvent, which prevents the release of the drug in gastric fluid.

2. The composition according to claim 1 wherein the coating further provides sustained release.

3. The composition according to claim 1 wherein the coating further provides site-specific release.

4. The composition according to claim 1 wherein the anionic water insoluble hydrophilic polymer is selected from the group consisting of polyacrylic acids, gums, alginates, pectins and their metallic salts.

5. The composition according to claim 4 wherein polyacrylic acid is selected from the group consisting of carboxyvinyl polymer, carbopol and polycarbofil.

6. The composition according to claim 4 wherein gums are selected from the group consisting of guar gum, xanthum gum, tragacanth gum, carragenan and locust bean gum.

7. The composition according to claim 1 wherein the water insoluble hydrophobic film forming polymer is selected from the group consisting of cellulose ether, shellac, zein, and waxes.

8. The composition according to claim 1 wherein the ratio of anionic water insoluble hydrophilic polymer to water insoluble hydrophobic film forming polymer is between 1:9 to 9:1.

9. The composition according to claim 1 wherein the coating composition further comprises plasticizers, pigments, colorants, antifoam agents, antioxidants, waxes, monoglycerides, emulsifiers, surfactants and other additives.

10. The composition according to claim 9 wherein the plasticizer may be selected from the group consisting of dibutyl phthalate, diethyl phthalate, dimethyl phthalate, benzyl benzoate, butyl and glycol esters of fatty acids, refined mineral oils, oleic acid, stearic acid, cetyl alcohol, stearyl alcohol, castor oil, corn oil, camphor, and mixtures thereof.

11. The composition according to claim 1 wherein the oral pharmaceutical composition is a tablet, bead, granule, or a capsule.

12. The composition according to claim 1 wherein the core may be a matrix tablet or a capsule containing the drug or pellets of the drug or pellets of the drug layered on a core material or microcapsules containing the drug material.
